(19) United States
(12) Patent Application Publication
(10) Pub. No.: US 2013/0071349 A1
(43) Pub. Date: Mar. 21, 2013

(54) BIODEGRADABLE POLYMERS FOR LOWERING INTRAOCULAR PRESSURE

(71) Applicants: Michael R. Robinson, Irvine, CA (US); Susan S. Lee, Los Angeles, CA (US); Scott M. Whitcup, Laguna Hills, CA (US)

(72) Inventors: Michael R. Robinson, Irvine, CA (US); Susan S. Lee, Los Angeles, CA (US); Scott M. Whitcup, Laguna Hills, CA (US)

(73) Assignee: ALLERGAN, INC., Irvine, CA (US)

(21) Appl. No.: 13/516,159

(22) PCT Filed: Mar. 1, 2011
(86) PCT No.: PCT/US11/26670

§ 371 Date: Jun. 14, 2012

Related U.S. Application Data
(60) Provisional application No. 61/309,648, filed on Mar. 2, 2010.

(51) Int. Cl. A61K 31/765; A61K 47/34

(52) U.S. Cl. CPC A61K 31/765
USPC 424/78.37

(57) ABSTRACT

The present invention provides a method of treating glaucoma, the method comprising the step of placing a polymer in an eye of a patient, which biologically degrades over a period of time to release biodegradants, which are effective to lower the intraocular pressure of the patient, thereby treating glaucoma. Said polymer is preferably selected from the group consisting of polymers of lactic acid, glycolic acid and/or mixtures thereof. More preferably the polymer is a copolymer of lactic acid and glycolic acid, e.g. a copolymer comprising from 50 to 100% lactic acid and from 0 to 50% glycolic acid, by weight.

### CROSS-REFERENCE

This application claims the benefit of U.S. Provisional Patent Application Ser. No. 61/309,648, filed on Mar. 2, 2010, the entire disclosure of which is incorporated herein by this specific reference.

### BACKGROUND OF THE INVENTION

1. Field of the Invention.

The present invention relates to the field of solid implants for ophthalmic use.

2. Summary of the Related Art

Glaucoma is a family of diseases commonly characterized by progressive optic neuropathy with associated visual field defects and is the leading cause of irreversible blindness in the world. Glaucoma is classified 3 broad headings: developmental, angle-closure, and open angle glaucoma (OAG). Open angle glaucoma is further categorized into primary OAG (POAG) and secondary OAG (includes pigmentary, pseudoexfoliation), the former being the predominant form of OAG. POAG is characterized as a multi-factorial optic neuropathy with a ‘characteristic acquired atrophy of the optic nerve and loss of ganglion cells and their axons’ developing in the presence of open anterior chamber angles, and manifesting characteristic visual field abnormalities. It is estimated that approximately 66.8 million people worldwide are affected with OAG of whom 6.7 million will progress to blindness in both eyes. It is estimated that 2.25 million people in the United States (US) over the age of 40 years have POAG, half of whom are unaware of their disease despite demonstrable visual field loss. Another 10 million persons in the US are estimated to have intraocular pressures (IOPs) greater than 21 mm Hg, or other risk factors for developing OAG; approximately 10% of these eyes will convert to OAG over the course of a decade.

Patient non-adherence to topical therapy is one of the major challenges to preventing vision loss due to glaucoma, as consistent IOP reduction is associated with reduced risks of developing and progressing optic nerve damage. Patients that take no medication are at the highest risk of vision loss from glaucoma, however, patients that intermittently take their medications are also at risk since IOP fluctuation has also been identified as an important risk factor for progression. There are a number of causes of non-adherence to glaucoma therapy, including the medication regimen and patient factors. Most glaucoma patients are elderly, and many have inherent difficulties taking medications, such as hearing difficulty, health literacy, physical or cognitive disability, as well as impaired visual acuity. As greater than 50% of glaucoma and patients with ocular hypertension (OHT) or elevated IOP are non-adherent to topical pharmacological therapy, improvements must be made to increase adherence, and thereby, improve visual outcomes for glaucoma and OHT patients. In patients that are non-adherent to medical therapy, guidelines are provided to clinicians assisting patients to be adherent, nevertheless, not infrequently, patients with OAG or OHT not taking their medication will require filtering surgery to control the IOP. The disadvantage of performing filtering surgery in patients with OAG or OHT are the significant sight-threatening complications that can occur with surgery. These include problems associated with retrobulbar anesthesia such as perforation of the globe, suprachoroidal hemorrhage, hypotony maculopathy, corneal decompensation, and cataract formation or progression of a pre-existing cataract. Post-operative endophthalmitis is major complication of any incisional surgery into the eye; however, the incidence has been dramatically reduced with the use of povidone iodine used topically pre-operatively. Given the risks associated with filtering surgery, sustained-release formulations releasing anti-hypertensive drugs are in development as an alternative to the management of elevated IOP.

Sustained-release drug delivery systems comprising bimatoprost (Bimatoprost Intracarneral Drug Delivery System or Bimatoprost IC DDS) which reduce the patient dependence on taking topical ocular anti-hypertensive medications to control the IOP have been described. (See Published United States Patent Application Serial No. 2005/0244464.). The Bimatoprost IC DDS refers to the implant itself which is pre-loaded in the applicator. This sustained-release implant uses a synthetic aliphatic polyester platform and delivers preservative-free Bimatoprost in the intracameral space (i.e. anterior chamber of the eye) for at least 3 months to control elevated IOP. The implants were designed to release from 10 to 40 μg of preservative-free Bimatoprost continuously over at least 3 months. In contrast, a 35 μl drop of LUMIGAN® ophthalmic solution (0.03% Bimatoprost solution), typical of what is used clinically, contains 10 μg of Bimatoprost. With topical therapy, a patient would have drug exposure to the surface of the eye totaling ˜900 μg over a 3-month period. Reducing the total daily drug exposure to the eye by ˜20 fold with the Bimatoprost IC DDS implant, and delivering the drug directly to the aqueous humor avoiding the eyelids and conjunctiva, reduces any adverse effects observed with topical LUMIGAN®. In addition, the continuous release of drug in the aqueous humor using the implant may reduce peak and trough drug levels in the aqueous humor that occurs with topical therapy. Since IOP variation appears to be an independent risk factor for glaucomatous damage establishing steady-state concentrations in the aqueous humor with the implant has the potential to establish lower fluctuation of the IOP's over a 24-hour period.

The Bimatoprost IC DDS is an intracameral sustained release drug implant that provides continuous release avoiding the peak and trough drug levels that occur in the aqueous humor with topical dosing. The steady state drug concentrations achieved in the aqueous humor with the implant can significantly lower the IOP fluctuation during the day and night. The implant is made of polymeric materials to provide maximal approximation of the implant to the iridocorneal angle. In addition, the size of the implant, which ranges from a diameter of approximately 0.1 to 1 mm, and lengths from 0.1 to 6 mm, enables the implant to be inserted into the anterior chamber using an applicator with a small gauge needle ranging from 22 to 30 G. Implant materials can be any combination of lactic acid and/or glycolic acid, as a homopolymer or a copolymer, that provides for sustained-release of drug into the outflow systems over time.

Bimatoprost IC DDS is injected into the anterior chamber near the corneal limbus through a 25 to 30-gauge needle with the applicator system. The polymer matrix slowly degrades so that there is no need to remove the implant once the drug has been released. The drug is expected to release over a 3 to 6 month period and the polymer matrix degradation is expected to be completed by 12 to 18 months. The polymer matrix used to manufacture the Bimatoprost IC DDS is a synthetic aliphatic polyester, i.e. a polymer of lactic acid and/or glycolic acid, and includes poly-(D,L-lactide) (PLA), polyglycolic acid (PGA), and the copolymer poly-(D, L, -lactide-co-glycolide) (PLGA). The PLGA and PLA polymers are known to degrade via backbone hydrolysis (bulk erosion) and the final degradation products of PLA and PLGA are lactic and glycolic acids which are non-toxic and considered natural metabolic compounds. Lactic and glycolic acids are eliminated safely via the Krebs' cycle by conversion to carbon dioxide and water. The PLA/PLGA polymers are from the Resomer product line available from Boehringer Ingelheim in Ingelheim, Germany.

PLGA is synthesized by means of random ring-opening co-polymerization of the cyclic dimers of glycolic acid and lactic acid. Successive monomeric units of glycolic or lactic acid are linked together in PLGA during polymerization by ester linkages. The ratio of lactide to glycolide used for the polymerization can be varied and this will alter the biodegradation characteristics of the product. It is possible to tailor the polymer degradation time by altering the ratio of the lactic acid and glycolic acid used during synthesis. Importantly, the rate of PLGA biodegradation, molecular weight and degree of crystallinity affects the drug release characteristics of drug delivery systems, thus giving polymer composition a significant role in the customization of implant characteristics.

The rate of drug release from biodegradable devices depends on the total surface area of the device, the percentage of loaded drug, the water solubility of drug, and the speed of polymer degradation. An advantage of PLGA-based delivery systems is that the rate and degree of drug release can be manipulated by altering the polymer composition to influence the degradation characteristics. The 3 main factors that determine the degradation rate of PLGA copolymers are the lactide:glycolide ratio, the lactide stereoisomeric composition (i.e., the amount of L- vs DL-lactide), and molecular weight. The lactide:glycolide ratio and stereoisomeric composition are most important for PLGA degradation as they determine polymer hydrophilicity and crystallinity. PLGA with a 1:1 ratio of lactic acid to glycolic acid degrades faster than PLA or PGA, and the degradation rate can be decreased by increasing the content of either lactide or glycolide. Polymers with degradation times ranging from weeks to years can be manufactured simply by customizing the lactide:glycolide ratio and lactide stereoisomeric composition. The versatility of PGA, PLA, and PLGA allows for construction of delivery systems to tailor the drug release for treating a variety of front and back of the eye diseases.

Drug release from PLA- and PLGA-based matrix drug delivery systems generally follows pseudo first-order or square root kinetics. Release is influenced by many factors including polymer, drug load, implant morphology, porosity, tortuosity, and deviation from sink conditions just to name a few. In general, release occurs in 3 phases: an initial burst release of drug from the surface, followed by a period of diffusional release which is governed by the inherent dissolution of drug, diffusion through internal pores into the surrounding media, and lastly, drug release associated with biodegradation of the polymer matrix. The rapid achievement of high drug concentrations followed by a longer period of continuous lower-dose release makes such delivery systems ideally suited for acute-onset diseases that require a loading dose of drug followed by tapering doses over a 1-day to 3-month period. More recent advancements in PLGA-based drug delivery systems have allowed for biphasic release characteristics with an initial high (burst) rate of drug release followed by sustained zero-order kinetic release (i.e., drug release rate from matrix is steady and independent of the drug concentration in the surrounding milieu) over longer periods. In addition, when desired for treating chronic diseases such as elevated IOP, these drug delivery systems can be designed to have steady state release following zero order kinetics from the onset.

PLA, PGA, and PLGA are cleaved predominantly by non-enzymatic hydrolysis of its ester linkages throughout the matrix, in the presence of water in the surrounding tissues. PLA, PGA, and PLGA polymers are biocompatible because they undergo hydrolysis in the body to produce the original monomers, lactic acid and/or glycolic acid. Lactic and glycolic acids are nontoxic and eliminated safely via the Krebs cycle by conversion to carbon dioxide and water. The biocompatibility of PLA, PGA and PLGA polymers has been further examined in both nonocular and ocular tissues of animals and humans. The findings indicate that the polymers are well tolerated.

### BRIEF SUMMARY OF THE INVENTION

Unexpectedly, during the investigation of sustained-release PLA, PGA, and PLGA polymer implants releasing bimatoprost in animal models, it was noted that the control implant which is polymer, alone, (i.e. PLA, PGA, and/or PLGA polymer implants without drug) lowered the intraocular pressure starting after approximately 1 to 2 months post-injection in animal models. While not wishing to be bound by theory, it is believed that the latent IOP reduction response occurs after a critical amount of biodegradation occurs, liberating polymer degradants that have the ability to lower the IOP. (See FIG. 1).

This invention provides a method of treating glaucoma and/or elevated intraocular pressure (IOP), the method comprising the step of placing a polymer in an eye of a patient, which polymer biologically degrades over a period of time to release biodegradants, which biodegradants are effective to lower the intraocular pressure of the patient, thereby treating and/or elevated IOP.

In one aspect of the invention the polymer is selected from the group consisting of polymers of lactic acid, glycolic acid and mixtures thereof, e.g. the polymer may be a lactic acid homopolymer or a glycolic acid homopolymer or a copolymer of lactic acid and glycolic acid, e.g. poly-(D,L-lactide) (PLA), polyglycolic acid (PGA), and the copolymer poly-(D, L, -lactide-co-glycolide) (PLGA).

In another aspect of the invention, there is provided a drug delivery system in the form of a first intraocular implant comprising an active pharmaceutical ingredient, e.g. bimatoprost, the active pharmaceutical ingredient being effective to lower the intraocular pressure of a patient having elevated intraocular pressure, wherein the active pharmaceutical ingredient is associated with a polymer that releases the active pharmaceutical ingredient into the eye of the patient over a period of time, and a second intraocular implant free of any the active pharmaceutical ingredient, wherein the second intraocular implant comprises a biodegradable polymer, which biologically degrades over a period of time to release biodegradants which are effective to lower the intraocular pressure of the patient.

### BRIEF DESCRIPTION OF THE DRAWINGS

FIG. 1 shows the biodegradation phases that the implants of the invention cycle through after placement in the eye.

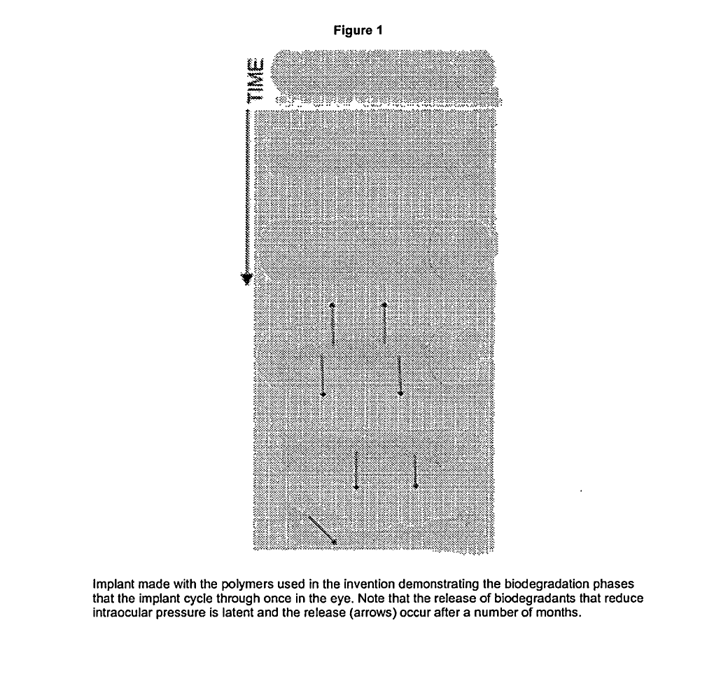

FIG. 2 shows the in-vitro release rate of a implant comprising bimatoprost in a PLGA polymer matrix.

FIG. 3 shows the mean differences in IOP between the treated, right eyes and the untreated, left eyes in various groups as the percentage of change from baseline (average values from Days −7 and −5).

FIG. 4 shows the IOP reduction from the polymer only implants (group 2) and Group 5 (30 ug) implants.

FIG. 5 is a photograph showing the bioerosion physical characteristics of the implants.

FIG. 6 is a photograph showing the internal excavation of the polymer, only, implants occurring during the bioerosion process.

FIG. 7 shows that IOP reduction occurs at all dose levels starting at approximately 2 months post-injection.

FIG. 8 shows that IOP reduction from baseline with the bimatoprost 30 ug implant ranges between 20 to 30% for approximately 3 months.

### DETAILED DESCRIPTION OF THE INVENTION

The following terms are defined as follows:

The term “associated with” means mixed with, dispersed within, coupled to, covering, or surrounding.

The term “API” means active pharmaceutical ingredient, including but not limited to those drugs listed in the Orange Book of the Food and Drug Administration.

The term “biodegradable polymer” refers to a polymer or polymers which degrade in vivo, and wherein erosion of the polymer or polymers over time occurs concurrent with or subsequent to release of the therapeutic agent. The terms “biodegradable” and “bioerodible” are equivalent and are used interchangeably herein. A biodegradable polymer may be a homopolymer, a copolymer, or a polymer comprising more than two different polymeric units.

The term “treat”, “treating”, or “treatment” as used herein, refers to reduction or resolution or prevention of an ocular condition, ocular injury or damage, or to promote healing of injured or damaged ocular tissue. A treatment is usually effective to reduce at least one symptom of an ocular condition, ocular injury or damage.

The term “effective” as used herein, refers to the level or amount of an agent, e.g. an API, needed to treat an ocular condition, or reduce or prevent ocular injury or damage without causing significant negative or adverse side effects to the eye or a region of the eye. In view of the above, an effective amount of a therapeutic agent, such as a prostamide or prostamide derivative or a biodegradant, is an amount that is effective in reducing at least one symptom of an ocular condition, e.g. elevated IO.

It has surprisingly been discovered that in a method of treating glaucoma and/or elevated IOP, the method comprising the step of placing a polymer in an eye of a patient, the polymer, itself, i.e. in the absence of an active pharmaceutical ingredient, biologically degrades in the eye over a period of time to release biodegradants, which biodegradants are effective to lower the intraocular pressure of the patient and thereby treat glaucoma and/or elevated IOP.

The polymer may be selected from the group consisting of polymers of lactic acid, glycolic acid and mixtures of lactic acid and glycolic acid, e.g. poly-(D,L-lactide) (PLA), polyglycolic acid (PGA), and the copolymer poly-(D, L, -lactide-co-glycolide) (PLGA). Preferably, the polymer is a copolymer of lactic acid and glycolic acid, i.e. the copolymer poly-(D, L, -lactide-co-glycolide) (PLGA).

The following Resomer products may be used as the second intraocular implant in the method and system of the present invention:

| Resomer | Monomer ratio | i.v. dL/g |
|---|---|---|
| RG502, | 50:50 poly (D,L-lactide-co-glycolide) | 0.2 |
| RG502H, | 50:50 poly (D,L-lactide-co-glycolide) | 0.2 |
| RG503, | 50:50 poly (D,L-lactide-co-glycolide) | 0.4 |
| RG504, | 50:50 poly (D,L-lactide-co-glycolide) | 0.5 |
| RG505, | 50:50 poly (D,L-lactide-co-glycolide) | 0.7 |
| RG506, | 50:50 poly (D,L-lactide-co-glycolide) | 0.8 |
| RG752S, | 75:25 poly (D,L lactide-co-glycolide) | 0.16-0.24 |
| RG755, | 75:25 poly(D,L lactide-co-glycolide) | 0.6(40000) |
| RG756, | 75:25 poly(D,L lactide-co-glycolide) | 0.8 |
| RG858, | 85:15 poly (D,L-lactide-co-glycolide) | 1.4 |
| R202H, | poly (D,L-lactide) | 0.16-0.24 |
| R203S | poly (D,L-lactide) | 0.25-0.35 |
| R206. | poly (D,L-lactide); acid end | 0.2 |
| R104 | poly (D,L-lactide) | (3500) |

In one aspect of the invention there is provided a method of treating glaucoma and/or elevated IOP, the method consisting essentially of the step of placing a biodegradable polymer in an eye, which polymer degrades in the eye to provide biodegradants which are effective to lower IOP, thereby treating glaucoma and ocular hypertension

In a still further aspect of the invention there is provided a method of treating glaucoma and ocular hypertension, comprising placing in the eye of a patient a first intraocular implant comprising an active pharmaceutical ingredient, the active pharmaceutical ingredient being effective to lower the intraocular pressure of a patient having elevated intraocular pressure, wherein the active pharmaceutical ingredient is associated with a biodegradable polymer that releases the active pharmaceutical ingredient into the eye of the patient over a period of time, and placing in the eye of a patient a second intraocular implant, free of any the active pharmaceutical ingredient, wherein the second intraocular implant comprises a biodegradable polymer, which biologically degrades over a period of time to release biodegradants which are effective to lower the intraocular pressure of the patient, thereby treating glaucoma and/or ocular hypertension. Said first and second intraocular implant may be placed in the patients eye, simultaneously, e.g. in the form of an aggregate of micro spheres, wherein the active pharmaceutical ingredient is associated with a plurality of micro spheres which is separate from a plurality of micro spheres comprising the biodegradable polymer.

Preferably, the first ocular implant releases the active pharmaceutical ingredient in an amount effective to lower the intraocular pressure of a patient having elevated intraocular pressure of the eye of the patient over a first period of time, wherein the first period of time is from one (1) day to three (3) months from the insertion of the first ocular implant into the eye of the patient.

Preferably the second ocular implant biologically degrades to release biodegradants which are effective to lower the intraocular pressure of the patient, over a second period of time, wherein the second period of time is from two (2) to six (6) months after the insertion of the second intraocular implant into the patients eye. More preferably, the first and the second period of time do not overlap.

In one aspect, the active pharmaceutical ingredient comprises bimatoprost.

In another aspect of the present invention there is provided a drug delivery system in the form of a first intraocular implant comprising an active pharmaceutical ingredient, the active pharmaceutical ingredient being effective to lower the intraocular pressure of a patient having elevated intraocular pressure, wherein the active pharmaceutical ingredient is associated with a biodegradable polymer that releases the active pharmaceutical ingredient into the eye of the patient over a period of time, and a second intraocular implant free of any the active pharmaceutical ingredient, wherein the second intraocular implant comprises a biodegradable polymer, which biologically degrades over a period of time to release biodegradants which are effective to lower the intraocular pressure of the patient.

Preferably, the first intraocular implant is in the form of micro spheres and the second intraocular implant is in the form of micro spheres.

More preferably, the first intraocular implant and the second intraocular implant are an aggregated mixture.

Most preferably, the active pharmaceutical ingredient is bimatoprost, e.g. the Bimatoprost Intracameral Drug Delivery System or Bimatoprost IC DDS, described above, may be combined with a biodegradable polymer, which biologically degrades over a period of time to release biodegradants which are effective to lower the intraocular pressure of the patient to provide the drug delivery system of the present invention.

A preferred implant formulation for use as the Bimatoprost IC DDS in the method and system of the invention is API 30%, R203S 45%, R202H 20%, PEG 3350 5% or API 20%, R203S 45%, R202H 10%, RG752S 20%, PEG 3350 5%. The range of concentrations of the constituents that can be used in the preferred implant formulation are API 5 to 40%, R203S 10 to 60%, R202H 5 to 20%, RG752S 5 to 40%, PEG 3350 0 to 15.

Suitable active pharmaceutical ingredients for use in the practice of this invention may be found in the Orange Book published by the Food and Drug Administration which lists drugs approved for treating glaucoma and/or lowering IOP. In general, the active pharmaceutical ingredients (APIs) that can be used in this invention are prostaglandins, prostaglandin analogues, and prostamides. Other APIs not related to prostaglandins or prostamides can be used with the above first intraocular implant include beta-adrenergic receptor antagonists, alpha adrenergic receptor agonists, less-selective sympathomimetics, carbonic anhydrase inhibitors, rho-kinase inhibitors, vaptans, anecortave acetate and analogues, ethacrynic acid, cannabinoids, cholinergic agonists including direct acting cholinergic agonists (miotic agents, parasympathomimetics), chlolinesterase inhibitors, and calcium channel blockers.

Combinations of ocular anti-hypertensives, such as a beta blocker and a prostaglandin/prostamide analogue, can also be used in the delivery systems. Other APIs outside of the class of ocular hypotensive agents can be used with the above first intraocular implant to treat a variety of ocular conditions. For example, anti-VEGF and other anti-angiogenesis compounds can be used to treat neovascular glaucoma. Another example is the use of corticosteroids or calcineurin inhibitors that can be used to treat diseases such as uveitis and corneal transplant rejection. These implants can also be placed in the subconjunctival space and in the vitreous.

An ocular implant comprising bimatoprost that is suitable for use in the method of the present invention is disclosed in Patent Application Publication No. 2005/0244464, which is hereby incorporated by reference in its entirety.

The first and second ocular implants may also include one or more ingredients which are conventionally employed in compositions of the same general type. The following non-limiting examples illustrate certain aspects of the present invention. Each formulation set forth in the following examples is prepared by in a conventional manner.

### EXAMPLE 1

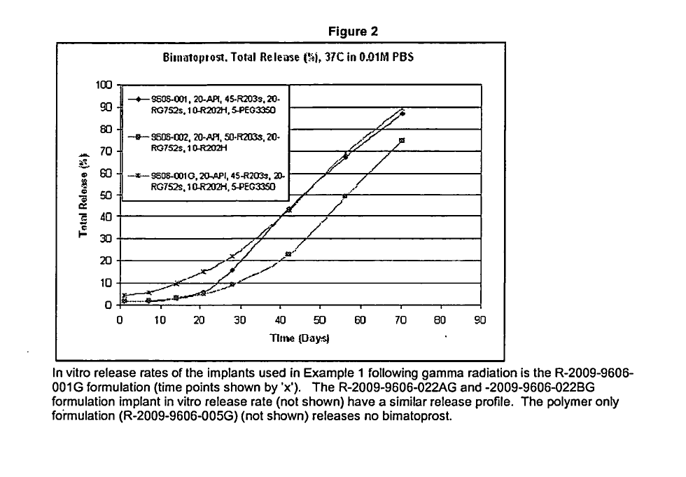

Forty eight purebred beagle dogs were dosed with sufficient amounts of Bimatoprost IC DDS implants with a composition: API 20%, R203S 45%, R202H 10%, RG752S 20%, and PEG 3350 5% to deliver 8, 15, 30 and 60 μg/day. The implants were manufactured using a hot melt extrusion process. This formulation that has an in vitro release rate demonstrating that the duration of drug release is over approximately 3 months (See FIG. 2). Polymer only (no bimatoprost) implants comprised 56.25% R203s, 25% RG752s, 12.25% R202H, 6.25% PEG-3350). Intracameral injections were performed using pre-loaded applicators without complications and the IOPs were monitored over time.

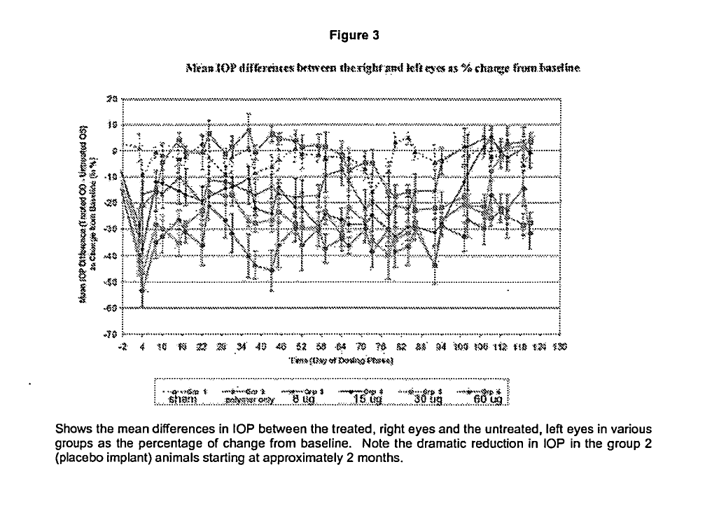

FIG. 3 shows the mean differences in IOP between the treated, right eyes and the untreated, left eyes in various groups as the percentage of change from baseline (average values from Days −7 and −5). All error bars represent standard errors. With groups 3, 4, 5, and 6 which received implants with bimatoprost, there was a significant IOP reduction compared with sham for approximately 3 months. By Day 112, the treated eyes of Groups 3, 4, and 5 no longer showed noticeable differences in IOP between the right, treated eyes and the left, untreated eyes and only the differences between the right, treated (polymer only implant) eyes and the left, untreated eyes of the Groups 2 and 6 animals remained noticeably different from baseline or the sham control Group 1. On Day 121, the differences in IOP between the right and left eyes of Group 2 (polymer only; 0 μg/eye) and Group 6 (60 μg/eye) were at −27.6% and −31.5% respectively.

The polymer only implant, unexpectedly, had a noticeable IOP lowering effect starting Day 78 and IOP in these eyes showed 27.6% lower than the contralateral untreated eyes on Day 121. By Day 112, the treated eyes with active implant Groups 3, 4, and 5 no longer showed noticeable differences in IOP between the right, treated eyes and the left, untreated eyes, while the Group 6 (60 μg/eye) treated had noticeably lowered IOP.

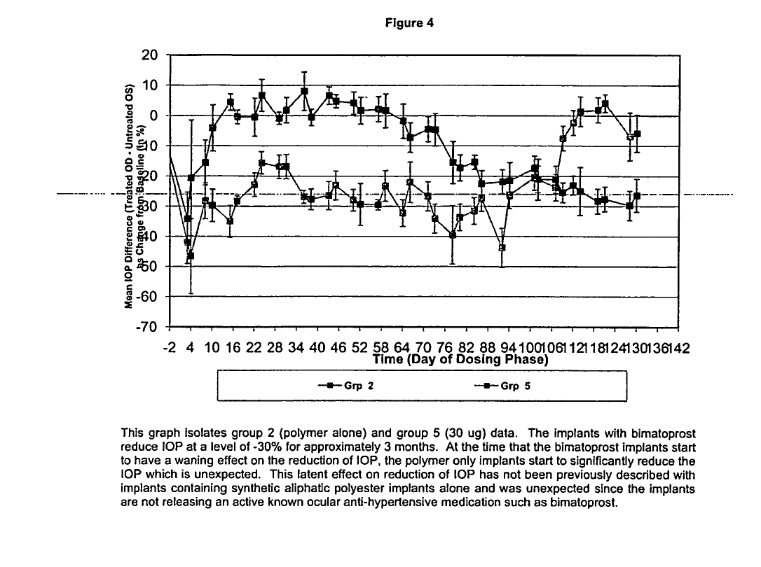

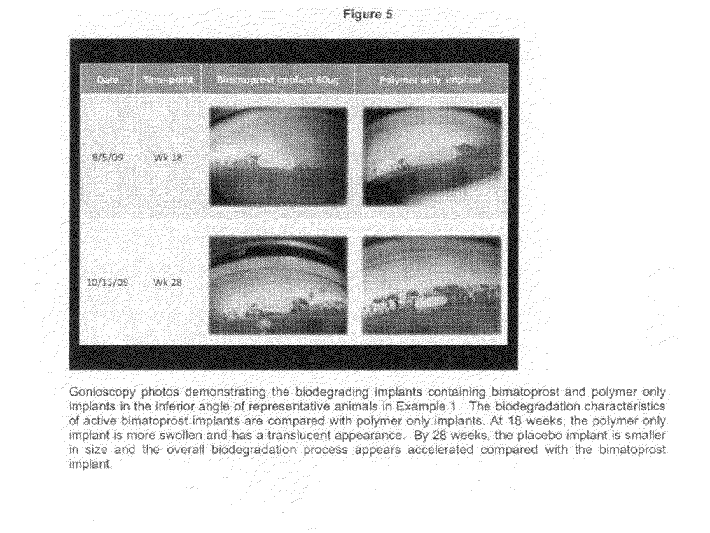

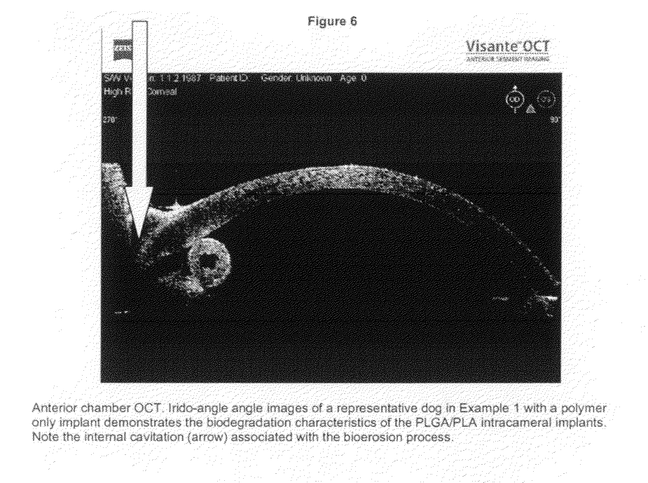

FIG. 4 shows the IOP reduction from the polymer only implants (group 2) and Group 5 (30 ug) implants. The conjunctival hyperemia produced by the polymer only implants was close to zero at most time points. The bioerosion physical characteristics of the implants are demonstrated in the photograph of FIG. 5. The biodegradation characteristics of active bimatoprost implants are compared with polymer only implants. At 18 weeks, the polymer only implant is more swollen and has a translucent appearance. By 28 weeks, the placebo implant is smaller in size and the overall biodegradation process appears accelerated compared with the bimatoprost implant. FIG. 6 demonstrates with anterior chamber OCT the internal excavation of the polymer only implants occurring during the bioerosion process.

Example 1 demonstrated that the polymer only implant has a latent effect at lowering IOP in dogs.

### EXAMPLE 2

A dose response study was conducted to determine if different size polymer only implants would show differences in reduction of IOP.

Twelve beagle dogs were dosed in 1 eye with polymer, only, implants (56.25% R203s, 25% RG752s, 12.25% R202H, 6.25% PEG-3350) of various sizes (volumes of 0.12, 0.20, and 0.30 mm³). The intracameral injections were performed using pre-loaded applicators with either 25- or 27 G needles.

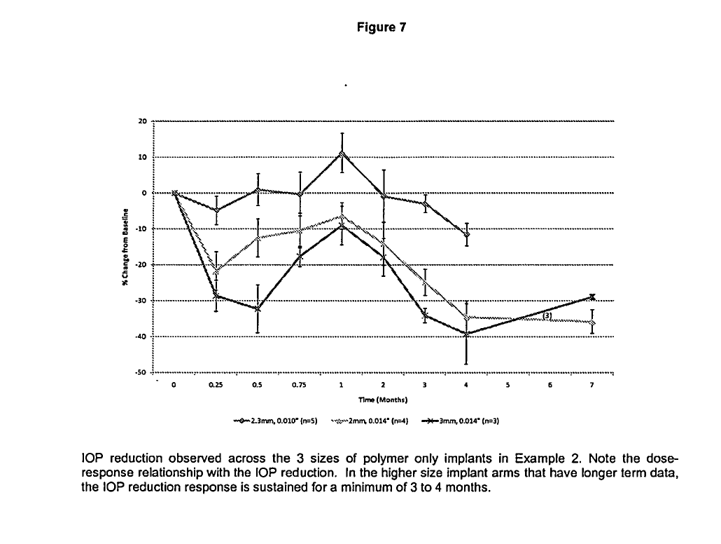

IOP reduction was demonstrated at all dose levels starting at approximately 2 months post-injection (FIG. 7). The IOP reduction from baseline values at 4 months was 12, 35, and 39% with the implants with volumes of 0.12, 0.20, and 0.30 mm³, respectively.

A dose-response relationship with regards to IOP reduction exists with synthetic aliphatic polyester implants without bimatoprost (i.e. polymer only implants).

### EXAMPLE 3

Polymer only implants study in an ocular hypertensive (OHT) monkey model.

12 cynomolgus monkeys had trabecular meshwork laser accomplished to achieve elevated IOP using standardized techniques. Six monkeys received an intracameral injection of a bimatoprost 30 ug implant. An additional 6 monkeys received a polymer only implant comprising 56.25% R203S, 25% RG752S, 12.25% R202H, 6.25% PEG-3350).

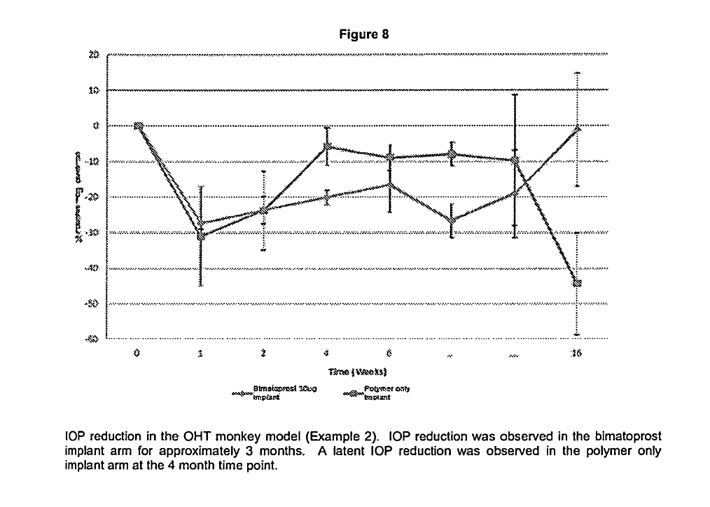

IOP reduction from baseline was demonstrated with the bimatoprost 30 ug implant ranging between 20 to 30% for approximately 3 months (See FIG. 8). Starting at approximately 3 months post-injection, the polymer only implants reduced the IOP by approximately 40% from baseline.

IOP reduction was observed in the OHT monkey exists with synthetic aliphatic polyester implants without bimatoprost (i.e. polymer, only, implants).

In summary, polymer only implants containing synthetic aliphatic polyester polymers have a latent IOP reduction potential when placed into the eye. The conclusions from the Examples described herein were that the polymer only implant appears to be liberating a degradant during the bioerosion process that is effective at lowering IOP after 1 to 2 months of being in the eye, preferably the anterior chamber. Once the IOP reduction occurs, it may persist for months thereafter. While not being wishing to be limited by theory, this active degradant may be an oligomer liberated from random scission of PLGA or PLA chains occurring during the bioerosion process that has receptor binding at the level of the trabecular meshwork (i.e. conventional outflow channels) or the anterior ciliary muscle (i.e. uvcoscleral flow or non-conventional outflow channels) that can facilitate aqueous outflow. The mechanism of action may also involve a reduction in the episcleral venous pressure that can allow for a reduction in the IOP. This polymer degradant can also be lactic acid, glycolic acid, a specific length of a PLGA or PLA chain, or other unknown molecular species. The reduction of IOP may also be due to the acidification of the aqueous humor from the lactic and glycolic acid monomers being released during the bioerosion process. Any combination and different concentrations of the individual constituents of PLGA/PLA or PGA extruded into implants would have a similar effect at IOP lowering since they all have a common biodegradation pathway and would produce the active degradant. Other biodegradable polymers may produce the active degradant to lower the IOP such as polyorthoesters (POE), polyanhydrides (PAH), polyethylene glycol (PEG), polyethylene glycol-PLGA (PEG-PLGA), polycaprolactone (PCL), biodegradable polyurethanes (derived from PCL/PEG), glycolide-co-lactide-co-caprolactone (PGLC) copolymer, polymethylidene malonate (PMM), polypropylene fumarate (PPF), and poly-N-vinyl pyrrolidone (PVP). Lastly, biodegradable block copolymers that are based on aliphatic polyester or poly(ortho ester) and polyethylene glycol (PEG) blocks, including ReGel, can be used to produce degradants that lower the IOP.

Since the IOP reduction response is latent, and appears when the implants are well into the bioerosion process (as demonstrated by physical swelling and internal cavitation of the implant), there appears to be a critical time in the bioerosion process beyond which the active degradants are produced, released from the implant complex, and the IOP is reduced. This reduction in the IOP demonstrating a dose-response relationship suggests a drug-receptor relationship. IOP reduction with polymer only implants was demonstrated in both dogs and monkeys. The polymer only implants and implants containing a known active anti-hypertensive drug, such as those detailed in Example 1 that contain bimatoprost, can be injected into the eye at the same time. The net IOP reduction with this combination approach provides a continuous IOP reduction from the time of injection out to 6 months duration or longer. The polymer only and polymer containing a known API, can be co-extruded into one implant. These polymer only implants using synthetic aliphatic polyester polymers can be placed in different locations in the eye such as sub-Tenon's, intracameral, suprachoroidal, and intravitreal space.

### EXAMPLE 4

A method of treating glaucoma and/or ocular hypertension is contemplated, the method comprising placing in the eye of a patient a first intraocular implant comprising an active pharmaceutical ingredient, the active pharmaceutical ingredient being effective to lower the intraocular pressure of a patient having elevated intraocular pressure, wherein the active pharmaceutical ingredient is associated with a biodegradable polymer that releases the active pharmaceutical ingredient into the eye of the patient over a period of time, and placing in the eye of a patient a second intraocular implant free of any the active pharmaceutical ingredient, wherein the second intraocular implant comprises a biodegradable polymer, which biologically degrades over a period of time to release biodegradants which are effective to lower the intraocular pressure of the patient, thereby treating glaucoma and/or ocular hypertension.

The method may further comprise where the first ocular implant releases the active pharmaceutical ingredient in an amount effective to lower the intraocular pressure of a patient having elevated intraocular pressure into the eye of the patient over a first period of time, wherein the first period of time is from one day to three months following the insertion of the first ocular implant into the eye of a patient.

The method may further comprise where the second ocular implant biologically degrades to release biodegradants which are effective to lower the intraocular pressure of the patient over a second period of time, wherein the second period of time is from two (2) to six (6) months after the insertion of the second intraocular implant into the patients eye.

The method may further comprise where the first and the second period of time do not overlap.

The method may further comprise where the first and the second ocular implants are simultaneously placed in the eye of the patient.

The method may further comprise where the first and the second ocular implants are in the form of micro spheres.

The method may further comprise where the first and the second ocular implants are simultaneously placed in the eye of the patient as an aggregate.

The method may further comprise where the second ocular implant comprises a polymer selected from the group consisting of polymers of lactic acid, glycolic acid and mixtures thereof.

The method may further comprise where the polymer is a copolymer of lactic acid and glycolic acid.

The method may further comprise where the copolymer comprises from 50 to 100% lactic acid and from 0 to 50% glycolic acid, by weight.

The method may further comprise where the polymer additionally comprises polyethylene glycol.

The method may further comprise where second ocular implant biologically degrades to release biodegradants which are effective to lower the intraocular pressure of the patient over a second period of time, wherein the second period of time is from two (2) to six (6) months after the insertion of the second intraocular implant into the patients eye.

The method may further comprise where the polymer additionally comprises polyethylene glycol.

### EXAMPLE 5

A drug delivery system in the form of a first intraocular implant comprising an active pharmaceutical ingredient, the active pharmaceutical ingredient being effective to lower the intraocular pressure of a patient having elevated intraocular pressure, wherein the active pharmaceutical ingredient is associated with a biodegradable polymer that releases the active pharmaceutical ingredient into the eye of the patient over a period of time, and a second intraocular implant free of any the active pharmaceutical ingredient, wherein the second intraocular implant comprises a biodegradable polymer, which biologically degrades over a period of time to release biodegradants which are effective to lower the intraocular pressure of the patient.

The drug delivery system may further comprise where the second ocular implant comprises a polymer selected from the group consisting of polymers of lactic acid, glycolic acid and mixtures thereof.

The drug delivery system may further comprise where the polymer is a copolymer of lactic acid and glycolic acid.

The drug delivery system may further comprise where the copolymer comprises from 50 to 100% lactic acid and from 0 to 50% glycolic acid, by weight.

The drug delivery system may further comprise where the polymer additionally comprises polyethylene glycol.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

1. A method of treating glaucoma and/or ocular hypertension, the method comprising the step of placing a polymer in an eye of a patient, which biologically degrades over a period of time to release biodegradants, which are effective to lower the intraocular pressure of the patient, thereby treating glaucoma and/or ocular hypertension.

2. The method of claim 1 wherein the polymer is selected from the group consisting of polymers of lactic acid, glycolic acid and mixtures thereof.

3. The method of claim 2 wherein the polymer is a copolymer of lactic acid and glycolic acid.

4. The method of claim 2 wherein the polymer additionally comprises polyethylene glycol.

5. A method of treating glaucoma and/or ocular hypertension, the method consisting essentially of the step of placing a biodegradable polymer in an eye, thereby treating glaucoma and/or ocular hypertension

6. The method of claim 5 wherein the polymer is selected from the group consisting of lactic acid, glycolic acid and mixtures thereof.

7. The method of claim 6 wherein the polymer is a copolymer of lactic acid and glycolic acid.

8. The method of claim 7 wherein the copolymer comprises from 50 to 100%, by weight, lactic acid and from 0 to 50%, by weight, glycolic acid, by weight.

9. The method of claim 6 wherein the polymer additionally comprises polyethylene glycol.

10. A method of treating glaucoma and/or ocular hypertension, the method consisting of the step of placing a biodegradable polymer in an eye, thereby treating glaucoma and/or ocular hypertension

11. The method of claim 10 wherein the polymer is selected from the group consisting of lactic acid, glycolic acid and mixtures thereof.

12. The method of claim 11 wherein the polymer is a copolymer of lactic acid and glycolic acid.

13. The method of claim 12 wherein the copolymer comprises from 50 to 100%, by weight, lactic acid and from 0 to 50%, by weight, glycolic acid, by weight.

14. The method of claim 10 wherein the polymer additionally comprises polyethylene glycol.
